(19) United States
(12) Patent Application Publication
(10) Pub. No.: US 2021/0402633 A1
(43) Pub. Date: Dec. 30, 2021

(54) Axially Rotating Cutting System and Method for Cutting Medical Tubing

(71) Applicant: SYNEO, LLC, West Palm Beach, FL (US)

(72) Inventor: Kaden McDonald, St. George, UT (US)

(21) Appl. No.: 17/361,682

(22) Filed: Jun. 29, 2021

Related U.S. Application Data
(60) Provisional application No. 63/046,395, filed on Jun. 30, 2020.

(51) Int. Cl. B26D 1/36; A61M 25/00; B26D 3/00; B26D 7/00

(52) U.S. Cl. CPC B26D 1/36; A61M 25/0013; A61M 2207/10; B26D 7/0006; B26D 3/001

(57) ABSTRACT

A system and method for making cuts in a catheter or other medical tubing may include one or more blades connected to a blade housing. The blade housing may be attached to one or more mounting stems, and the mounting stems are coupled to a drive mechanism to axially rotate the blade housing and blade. A cradle plate is positioned proximal to the blade. The cradle plate includes a circular passageway extending therethrough non-parallel to the axis of rotation of the blade for holding medical tubing to be cut. The cradle plate also includes an arced cut-out in the axis of rotation of the blade to allow the blade to rotate into the arced cut-out to cut the tubing without colliding with the cradle plate.

### CROSS-REFERENCE TO RELATED APPLICATION

A claim for priority to the Jun. 30, 2020 filing date of U.S. Provisional Patent Application No. 63/046,395, titled AXIALLY ROTATING CUTTING SYSTEM AND METHOD FOR CUTTING MEDICAL TUBING (“the '395 Provisional application”), is hereby made pursuant to 35 U.S.C. § 119(e). The entire disclosure of the '395 Provisional application is hereby incorporated herein.

### TECHNICAL FIELD

This disclosure relates generally to cutting mechanisms to cut or skive catheters or other medical or surgical tubes. More specifically the disclosure relates to a system and method for cutting by use of a rotating axially moving tool to make lateral openings in catheter tubes.

### RELATED ART

Catheters are used in a variety of applications in the medical field. For example, catheters may be used for infusing drugs or nutrients into a patient's body, withdrawing bodily fluids from a patient's body drainage, administration of fluids or gases, access by surgical instruments, and also perform a wide variety of other tasks. Catheters are commonly used for infusing fluid, such as saline solution, various medicaments, and/or total parenteral nutrition, into a patient, withdrawing blood from a patient, and/or monitoring various parameters of the patient's vascular system. Depending on the function of the catheter, specialized materials, shapes, and openings may needed. For example, specialized catheters may include one or more of the following features, depending on the type of catheter: flares, flanges, angular welds, bonds, multi-lumen shape transitions, butt welds, balloon to catheter welds, neck downs, soft-tip fusion, metal-to-tube adhering, sheaths, dilators, radio-opaque, strain relief bonds, swaged metal needle guides, tube-in-tube bonds, etc. Drills may be used to form many specialized types of cuts for specialized features, but not all.

Forming these specialized catheters requires precise equipment. For example, if a catheter is cut with a standard razor or scissors, it often results in a blunt end that can present sharp edges that may scrape and traumatize the internal tissues within the patient's body. Edges and cuts on a catheter must be very uniform and cut smoothly. Any rough surfaces may scrape tissues, and/or encourage the accumulation of blood clots or bacteria. Many times drills are used to precision cut, however, there are limitations to the types of holes that may be formed by drills. For example, drills may not be used to form slots in tubing.

Thus, there is a need for a skiving mechanism that forms needed cuts in catheters or other medical tubing in a smooth, uniform, and easily controlled mechanism.

### SUMMARY

This disclosure, in at least one aspect, relates to a system for cutting a catheter. The system may include: a blade connected to a blade housing, the blade housing having a first end and a second end, and the blade having an axis of rotation; a first mounting stem having a distal end and a medial end, the distal end of the first mounting stem coupled to a drive mechanism, and the first end of the blade housing receivable in the medial end of the first mounting stem; a second mounting stem having a distal end and a medial end, the distal end of the second mounting stem coupled to the drive mechanism, and the second end of the blade housing receivable in the medial end of the second mounting stem; and a cradle plate positioned proximal to the blade, the cradle plate comprising a circular passageway extending therethrough non-parallel to the axis of rotation of the blade, and an arced cut-out in the axis of rotation of the blade, the blade receivable within the arced cut-out.

In some configurations, the medial end of the first mounting stem comprises a void sized to receive the first end of the blade housing. Similarly, the medial end of the second mounting stem may comprise a void sized to receive the second end of the blade housing.

According to another aspect, various types of blades may be used. For example, the blade may be a single vertical blade, a double horizontal blade, etc. The blade housing in some configurations is injection molded and the blade is integral to the blade housing. The cutting member may be disposable. In other configurations, the blade is not integral to the housing. The drive mechanism may comprise a coupled computer numerical controlled power train.

According to another aspect, a system for cutting a catheter may comprise: a cutting member connected to a cutting member housing, the cutting member having an axis of rotation; a mounting stem, the mounting stem coupled to a drive mechanism and the cutting member housing; and a cradle plate positioned proximal to the cutting member, the cradle plate comprising a passageway extending therethrough non-parallel to the axis of rotation of the cutting member, and a cut-out in the axis of rotation of the cutting member, the cut-out sized to accept the cutting member. In some configurations, the passageway comprises a round cross-section. The cut-out may have any shape or size desired, and in some configurations, the cut-out is an arced cut-out.

In some configurations, the system may further include a second mounting stem. The second mounting stem may be coupled to the drive mechanism and the second end of the cutting member housing, and the mounting stem coupled to the drive mechanism and the first end of the cutting member housing. The axis of rotation may be a vertical axis of rotation.

According to another aspect, a method for cutting a medical tube is described, and may include the steps of: selecting a cutting device, the cutting device comprising: a blade housing, a blade connected to the blade housing, the blade housing having a first end and a second end, and the blade having an axis of rotation; a first mounting stem coupled to the first end of the blade housing, a second mounting stem coupled to the second end of the blade housing; and a cradle plate positioned proximal to the blade, the cradle plate comprising a circular passageway extending therethrough perpendicular to the axis of rotation of the blade, and an arced cut-out in the axis of rotation of the blade, the blade receivable within the arced cut-out; coupling the medical tube to a gripper; positioning the medical tube within the circular passageway of the cradle plate; and cutting the medical tube by rotating the blade axially into the arced cut-out of the cradle plate.

In some configurations, the step of rotating the blade axially into the arced cut-out of the cradle plate comprises rotating the blade at least 180 degrees, the blade thereby cutting an arc-shaped cut out in the medical tube. In other configurations, the step of cutting the medical tubing by rotating the blade housing axially into the arced cut-out of the cradle plate comprises: rotating the blade into the arced cut-out; the gripper moving the medical tube a length; and rotating the blade out of the arced cut-out, the blade thereby cutting a slotted opening in the medical tube.

Other aspects, as well as features and advantages of various aspects of the disclosed subject matter will become apparent to one of ordinary skill in the art form the ensuing description, the accompanying drawings and the appended claims.

### BRIEF DESCRIPTION OF THE DRAWINGS

In the drawings:

FIG. 1 is a perspective view of a medical tubing cutting system;

FIG. 2 is a perspective view of the medical tubing cutting system of FIG. 1 with the window and frame removed;

FIG. 3 is a perspective view of a portion of the medical tubing cutting system of FIG. 2, including the blade, blade housing, and mounting stems;

FIG. 4 is an exploded view of FIG. 3;

FIG. 5 is a perspective view of one configuration of a blade and blade housing;

FIG. 6 is a perspective view of another configuration of a blade and blade housing;

FIG. 7 is a perspective view of a portion of the medical tubing cutting system, including the blade, blade housing, mounting stems, and cradle plate;

FIG. 8 is a cross-sectional view of FIG. 7;

FIG. 9 is a perspective view of a cradle plate;

FIG. 10 is a perspective view of a cutting system, including a gripper and a portion of medical tubing;

FIG. 11 is a perspective, close-up view of the blade as it is rotated into the arced-cut out of the cradle plate; and

FIG. 12 is a perspective, close-up view of a piece of medical tubing being cut by a dual-type blade.

### DETAILED DESCRIPTION

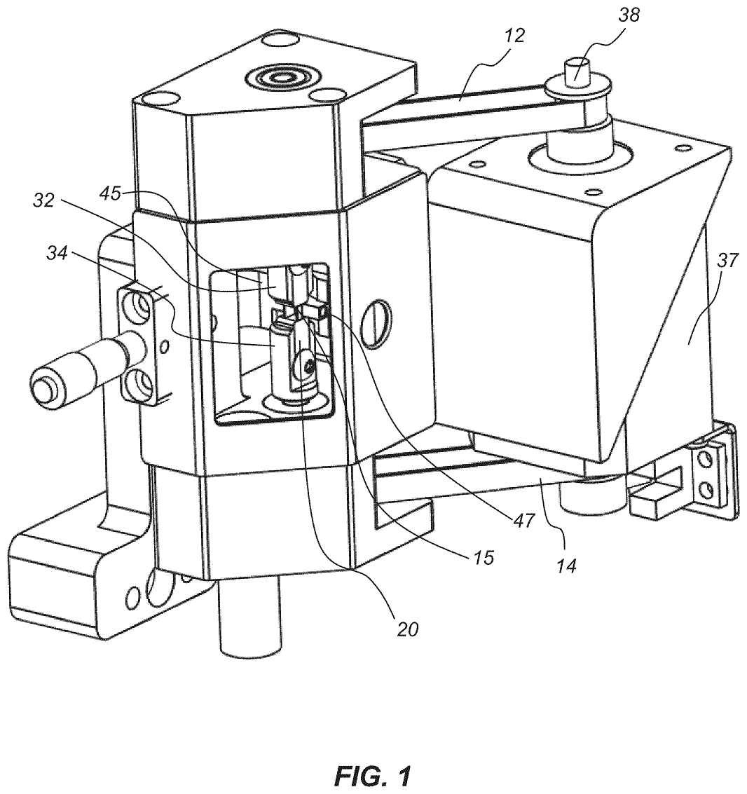

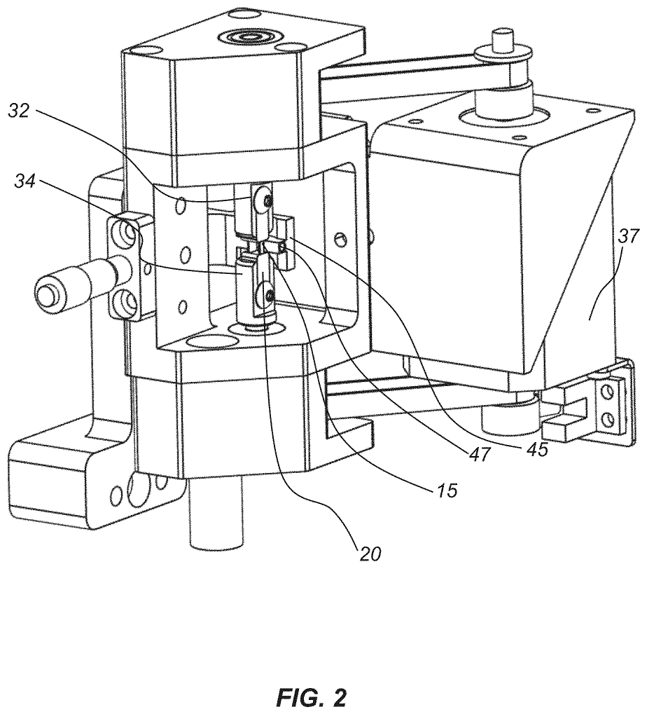

FIGS. 1-2 illustrate an embodiment of a system 10 for cutting a catheter or other medical tubing. As used herein, “catheter” or “tube” means any type of medical tubing, whether hollow or solid, and formed of any type of material. For example, thermoplastic tubing is common for medical tubing, and includes but is not limited to fluorinated ethylene propylene, ethylene tetrafluoroethylene, nylon, polycarbonate, polyether ether ketone, polyethylene terephthalate, polyamide; polyethylene, polyurethane, polypropylene, braided tubing, vinyl, polytetrafluoroethylene; polyvinyl carbonate. The system and methods described herein may be applied to any type of catheter, tube, etc.

The system may comprise a blade 15, which is rigidly or semi-rigidly held in a blade housing 20, and mounting stems 32, 34 that couple the blade housing 20 to a drive mechanism 37. A cradle plate 45 is positioned proximal to the blade 15, and includes a circular passageway 47 non-parallel to the axis of rotation of the blade 15 for a catheter or other medical tubing to be pulled through the cradle plate. The cradle plate is proximal to the blade such that the catheter or other tubing being cut is within the cutting pathway of the blade. In many configurations, the cradle plate 45 is positioned within a few millimeters of the blade, such that rotation of the blade may pass through one or more cut-outs in the cradle plate 45 as described below. The circular passageway 47 may be perpendicular or at an angle different than 90° that is non-parallel. Cradle plate 45 also includes and an arced cut-out 58 (not visible in FIGS. 1-2) positioned in the axis of rotation of the blade 15, so the blade 15 can rotate into the arced cut-out 58 to cut the catheter without colliding with the cradle plate 45 as described in more detail below. Mounting stems 32, 34 may be coupled to the drive mechanism 37 by any suitable method. For example, belts 12, 14 (FIG. 1) may couple the mounting stems to a drive shaft 38 of the drive mechanism 37. Any other suitable method may be provided to couple the one or more mounting stems to the drive mechanism 37.

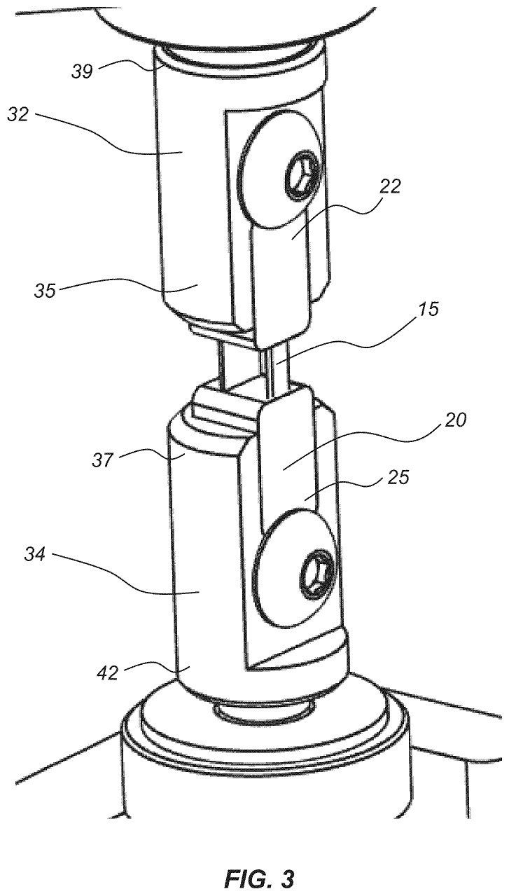

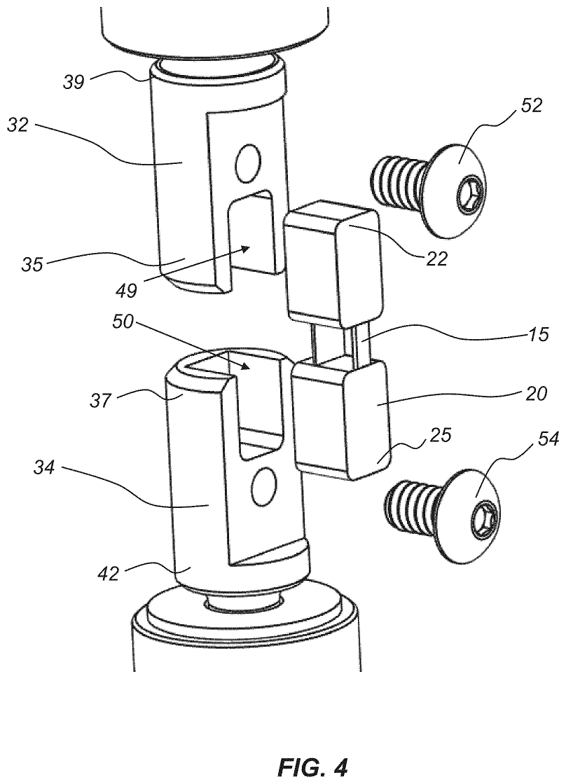

Turning now to FIGS. 3-4, the blade 15, blade housing 20, and mounting stems 32, 34 are shown (other portions of the system have been removed from these views for clarity). The blade 15 has a vertical axis of rotation, and is rotated through its axis of rotation by the drive mechanism 37. The blade 15 is coupled to the drive mechanism through the blade housing 20 and one or more of the mounting stems 32, 34. In some configurations, a single mounting stem may couple the blade housing 20 to the drive mechanism 37. In other configurations, two or more mounting stems may be used to couple the blade housing 20 to the drive mechanism 37. In the configuration shown, two mounting stems 32, 34 may be used to reduce torque on the blade housing 20. The blade housing 20 may have a first end 22 and a second end 25, and each end may be coupled to a mounting stem.

The one or more mounting stems may be coupled to the blade housing 20 through a press fit, frictional fit, snap fit or any other suitable means. The coupling may be permanent or removable. A removable connection may allow various types of blades to be used. In one configuration, mounting stems 32, 34 may each have a medial end (35 on the first mounting stem 32 and 36 on the second mounting stem 34) and a distal end (39 on the first mounting stem 32 and 42 on the second mounting stem 34). The medial end 35, 36 of the mounting stems 32, 34 may include a structure for coupling to the blade housing 20. For example, the medial end 35, 36 of the mounting stems 32, 34 may include a void sized to receive a portion of the blade housing 20. As seen in FIG. 4, the medial end 35 of the first mounting stem 32 includes a void 49 sized to receive the first end 22 of the blade housing 20. The medial end 36 of the second mounting stem 34 includes a void 50 sized to receive the second end 25. The blade housing 20 may be inserted with the first end 22 of the blade housing 20 in the void 49, and the second end 25 of the blade housing 20 in the void 50. The blade housing 20 may be further secured by any suitable means. For example, in FIG. 4, screws 52, 54 are provided to secure the blade housing 20 in place. Other types of connections or connecting mechanisms may be used to connect the blade housing 20 to the one or more mounting stems.

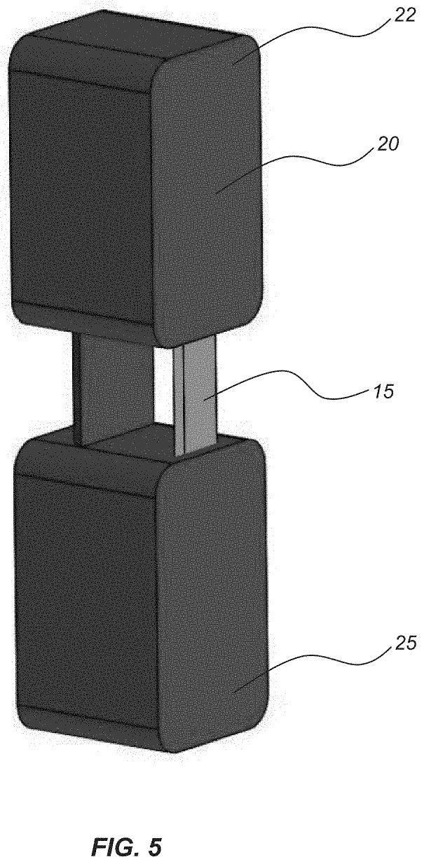

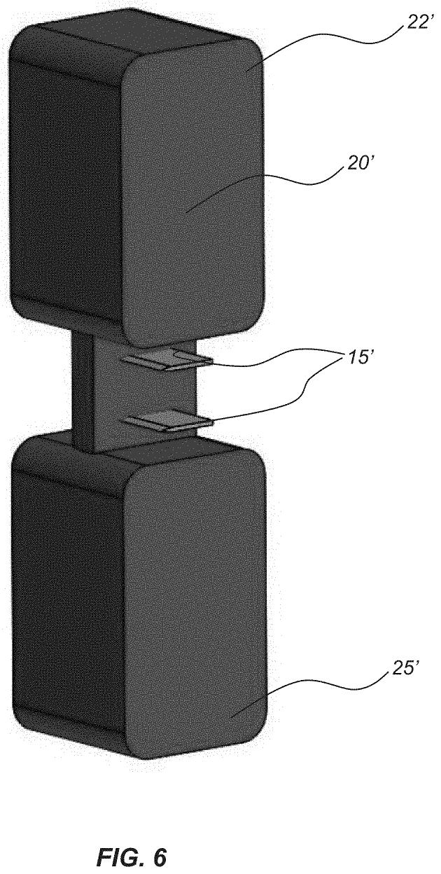

Turning now to FIGS. 5-6, two exemplary configurations of blade housings are shown (20 and 20′, respectively). The blade housing 20 may be any suitable shape or size and made from any suitable materials. In some configurations, the blade housing 20 is injection-molded. The blade 15 and/or the blade housing 20 may be disposable and/or consumable. In other configurations, the blade may be sharpened and/or replaced with a new blade as desired. The blade housing 20 may have a generally ovular or capsule-shape, or may have any other suitable shape depending on the application as needed. Because a sterile environment is often required for medical tubing, the blade housing 20 and/or blade 15 may be manufactured fairly inexpensively to be easily replaced.

FIG. 5 shows a blade housing 20 with a single vertical blade 15. FIG. 6 shows a blade housing 20′ with a dual blade 15′. A single vertical blade 15 may be used in applications where an arc or slot is desired, as described in more detail below. A dual blade 15′ may be used for applications where a slot between two holes is desired, again as described in more detail below. Other types of blades may also be used and are contemplated. Similarly, one, two, three, or more blades may be provided on a blade housing depending on the type of cut needed.

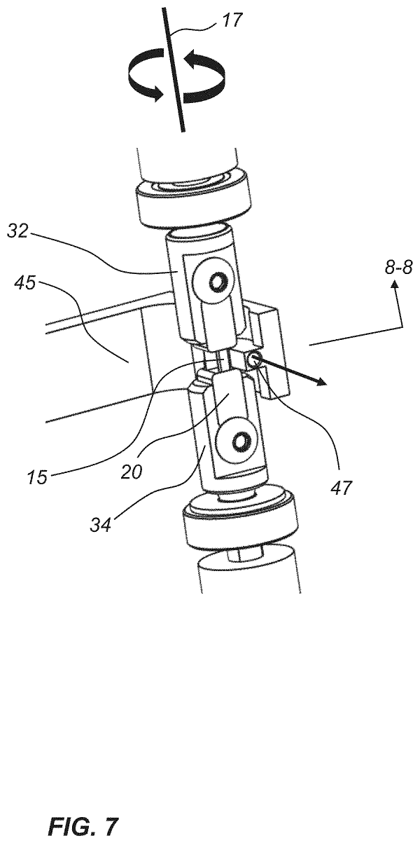

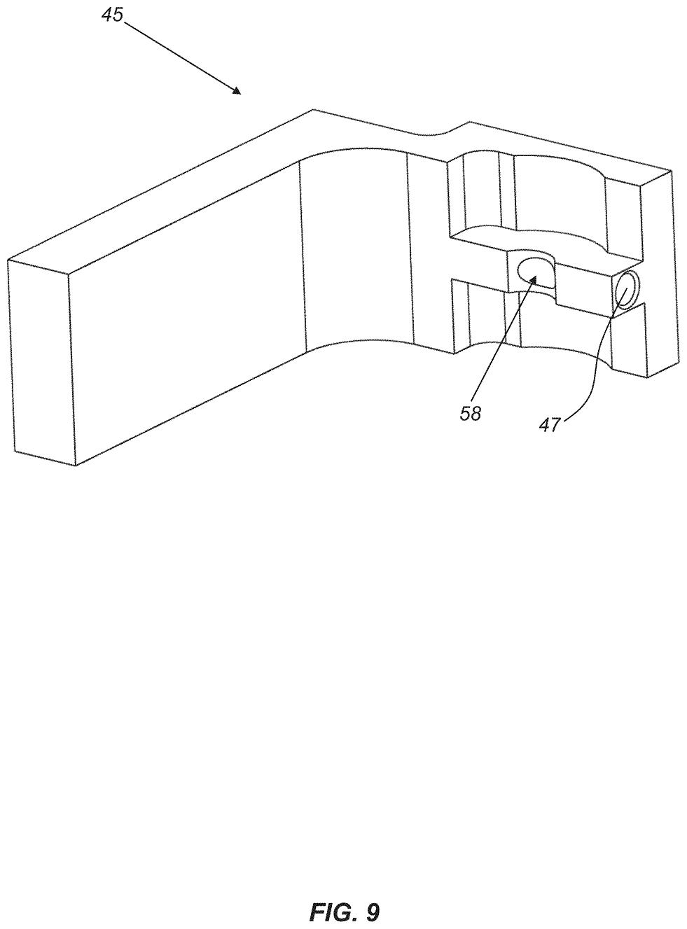

Turning now to FIGS. 7-9, the blade 15, blade housing 20, mounting stems 32, 34, and cradle plate 45 are shown. (The other portions of the system 10 have been removed from these views for clarity.) The blade has an axis of rotation, indicated at 17 in FIG. 7. The circular passageway 47 of the cradle block is non-parallel, and may be perpendicular, to the axis of rotation 17, and medical tubing is pulled through the circular passageway 47 as indicated at arrow 24. The medical tubing is generally covered by the cradle plate 45 and located within the circular passageway 47. An arced cut-out 58 (best seen in FIG. 9) may be provided in the circular passageway 47 of the cradle plate 45 in the axis of rotation of the blade. The medical tubing is exposed at this arced cut-out 58, creating an area for the blade 15 to rotate into the cradle plate 45 (and thus into any medical tubing located in the circular passageway 47) without colliding with the cradle plate 45.

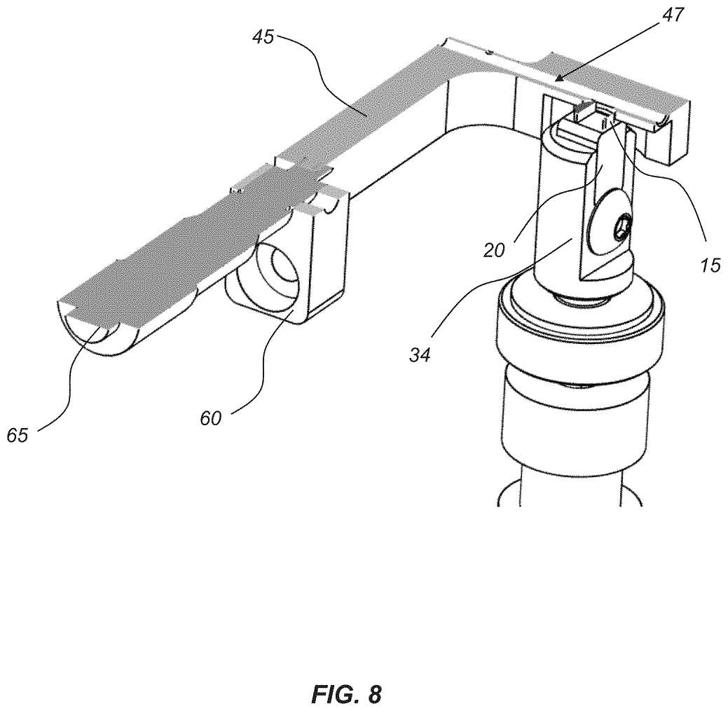

As the blade 15 is rotated through its axis of rotation 17 it cuts into the medical tubing. The depth at which the blade 15 cuts is carefully controlled, and the depth may be adjusted in a variety of ways according to any suitable known methods. In some configurations, a cradle plate depth adjustment block 60 (FIG. 8) is provided with a specific depth. Various depths of cradle plate blocks may be attached to the cradle plate to provide various depths of cuts. In some configurations, a cradle plate depth adjustment block 60 may be provided with a micrometer 65. Adjustment of the micrometer 65 adjusts the depth of the cradle plate. In other configurations, several depth adjustment blocks may be provided, each with a micrometer that is fixed at a specific depth. This may allow a user to switch out depth blocks to change the depth of the cut. For example, some applications may require a catheter that has a cut-out into the side, but not through to the lumen of the catheter, so a shallow depth may be required. In other configurations, a cut into the lumen may be required, so an increased depth may be required.

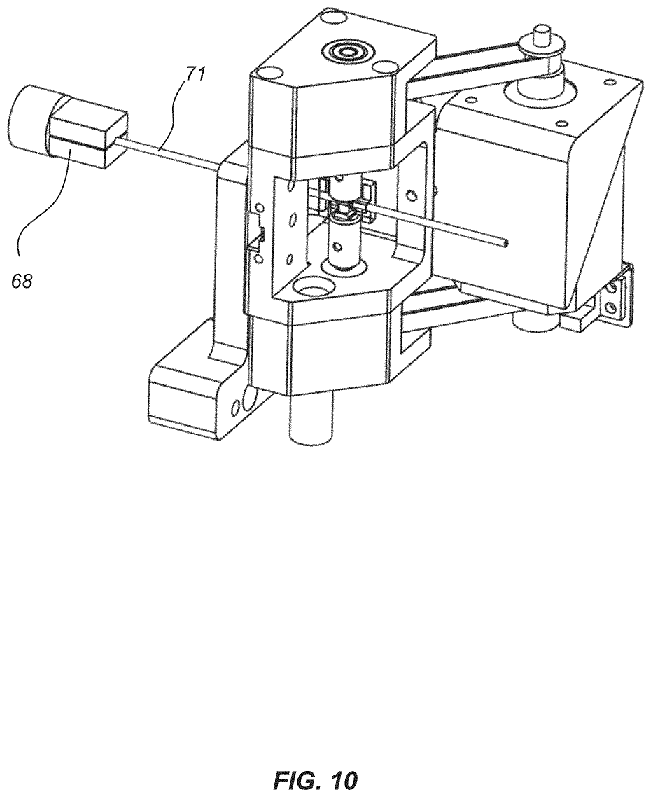

Similarly, the system may allow the length of the cut to be controlled. The length of the cut may be controlled by movement of the tubing. In some configurations, a gripper 68 (FIG. 10) may be provided (such as a CNC-positioned gripper) to move the tubing 71 forward and/or backward within the circular passageway 47. The gripper 68 may move the tubing 71 to the desired location, then the blade housing 20 may rotated about its vertical center axis causing the blade to cut into the tubing. If the cut desired is only an arc, then the blade housing is rotated further to exit the blade from the cradle plate completing the cut. If a slot cut is desired, the blade is first rotated to cut into the tubing then the tubing is pulled the desired slot length before stopping and allow the blade to finish the cut exiting the tubing.

The cradle plate 45 (see FIG. 9) may include the circular passageway 47, as well as the arced cut-out 58 with the circular passageway 47 the axis of rotation of the blade. As described above, any medical tubing locate within passageway 47 is exposed at the arced cut-out 58. It will be appreciated that while the passageway 47 is shown as being circular, any other suitable shape may be used, and the size of the passageway may also be larger or smaller depending on the medical tubing desired to be cut. Similarly, the arced cut-out 58 may have a different shape and/or size depending on the application for the cutting system 10.

In use, a user may first select a type of cut desired in a portion of tubing. Depending on the type of cut desired, the user may select a type of blade to be used to achieve the cut. The user may mount a blade housing having the desired type of blade onto the cutting system. For example, if one mounting stem is provided, the user may connect the blade housing to the mounting stem. If two mounting stems are provided, the user may connect a first end of the blade housing to a first mounting stem and the second end of the blade housing to the second mounting stem.

The user may then select the tubing, and position it within the circular passageway 47 and/or attach it to a gripper 68. In some configurations, the gripper 68 may position the tubing in the passageway 47. After the user has positioned the blade housing and the tubing 71, the system may provide automatic control over the equipment to ensure precise cutting (for example, through use of CNC). The blade housing may be rotated on its axis of rotation, into the arced cut-out and the medical tubing. The drive mechanism may provide the movement of the blade housing, with the mounting stems coupling the blade housing to the drive mechanism. If desired, the blade housing may be rotated through the arced cut-out around 180 degrees or more to create an arced cut-out within the medical tubing. Or, the blade housing may be rotated less than 180 degrees, for example, about 10 degrees to about 90 degrees (for an entry cut), and the gripper may then move the medical tubing within the circular passageway 47 for a specified length. The blade housing may then be rotated out of the arced cut-out (for an exit cut), creating a slot within the medical tubing.

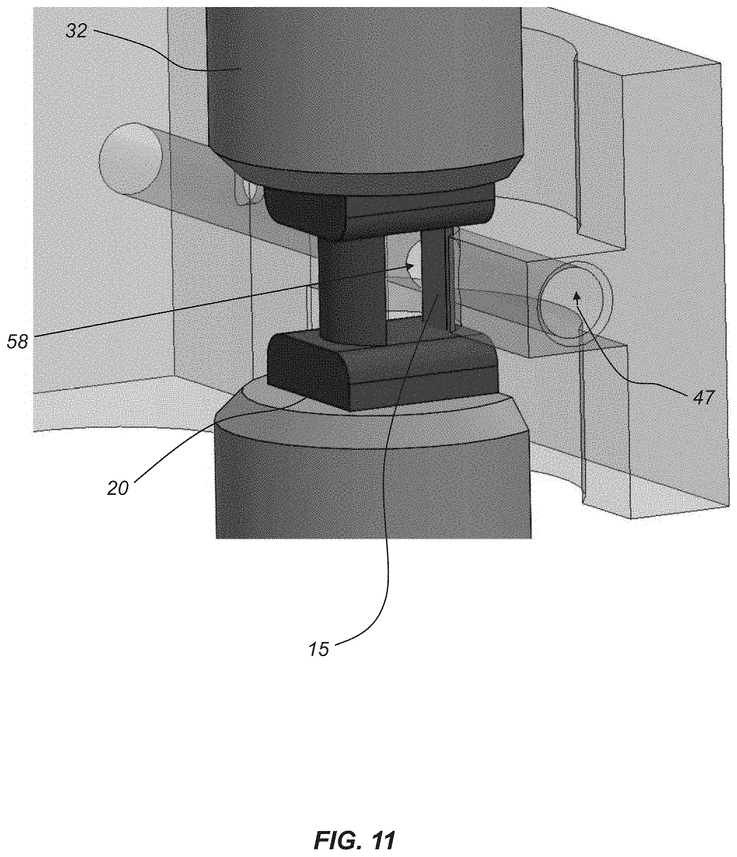

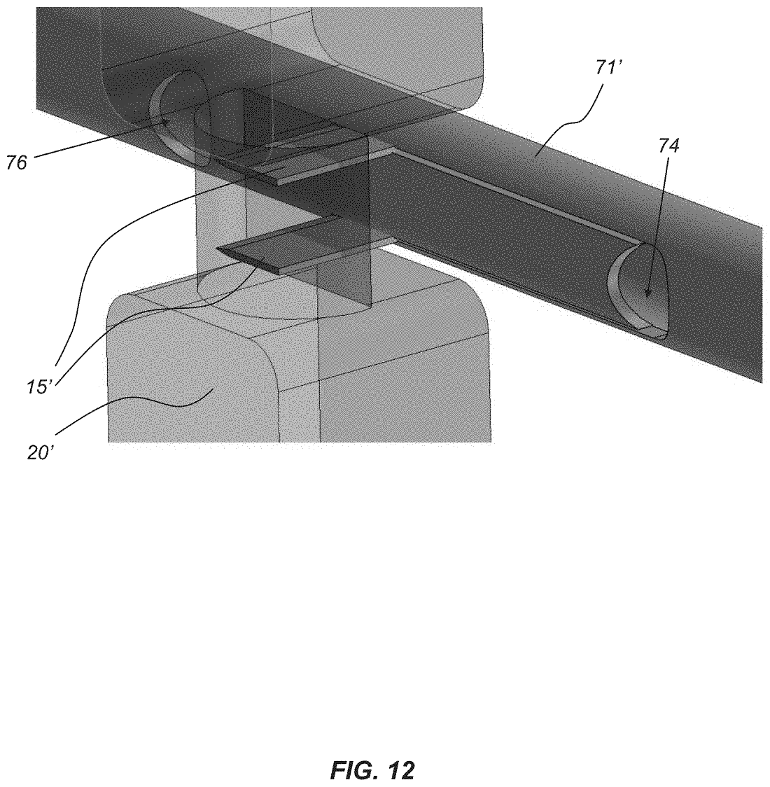

As shown in FIG. 11, a blade housing 20 with a single vertical blade may be rotated into the cut-out 58 of the cradle plate to cut any tubing that is located in the passageway 47. As shown in FIG. 12, a blade housing 20′ with dual blades 15′ may be used to cut a slot in tubing 71′ from a first hole 74 to a second hole 76. In some configurations, a single gripper may be provided to hold the medical tubing. In other configurations, one or more machine actuated clamps may hold parts of the tubing upstream and/or downstream from the blade housing to increase rigidity of the tubing during entry and exit cuts. Material cut from medical tubing may be removed via vacuum for small offcuts, and one or more grippers may be used to remove material for large offcuts.

It will be appreciated that a continuous flow of tubing may be utilized through the system 10. The tubing 71, 71′ may be cut to specified lengths within the same system 10 or other system. The action of cutting specific lengths of tubing may be upstream or downstream of the system 10.

Although the foregoing disclosure provides many specifics, such as use of the system to cut catheters, it will be appreciated that other medical or surgical tubes are contemplated and these should not be construed as limiting the scope of any of the ensuing claims. Other embodiments may be devised which do not depart from the scopes of the claims. Features from different embodiments may be employed separately or in combination. Accordingly, all additions, deletions and modifications to the disclosed subject matter that fall within the scopes of the claims are to be embraced thereby. The scope of each claim is indicated and limited only by its plain language and the full scope of available legal equivalents to its elements.

1. A system for cutting a catheter, comprising:
a blade connected to a blade housing, the blade housing having a first end and a second end, and the blade having an axis of rotation;
a first mounting stem having a distal end and a medial end, the distal end of the first mounting stem coupled to a drive mechanism, and the first end of the blade housing receivable in the medial end of the first mounting stem;
a second mounting stem having a distal end and a medial end, the distal end of the second mounting stem coupled to the drive mechanism, and the second end of the blade housing receivable in the medial end of the second mounting stem; and
a cradle plate positioned proximal to the blade, the cradle plate comprising a circular passageway extending therethrough non-parallel to the axis of rotation of the blade, the circular passageway for receiving the catheter, the cradle plate further comprising an arced cut-out in the axis of rotation of the blade, the blade receivable within the arced cut-out.

2. The system of claim 1, wherein the medial end of the first mounting stem comprises a void sized to receive the first end of the blade housing and wherein the medial end of the second mounting stem comprises a void sized to receive the second end of the blade housing.

3. The system according to claim 1, wherein the blade comprises at least one of a single vertical blade and a double horizontal blade.

4. The system according to claim 1, wherein the drive mechanism comprises a coupled computer numerical controlled power train.

5. The system according to claim 1, wherein blade housing is injection molded and the blade is integral to the blade housing.

6. A system for cutting a catheter, comprising:
a cutting member connected to a cutting member housing, the cutting member having an axis of rotation;
a mounting stem, the mounting stem coupled to a drive mechanism and the cutting member housing; and
a cradle plate positioned proximal to the cutting member, the cradle plate comprising a passageway extending therethrough non-parallel to the axis of rotation of the cutting member, and a cut-out in the axis of rotation of the cutting member, the cut-out sized to accept the cutting member.

7. The system of claim 6, wherein the passageway comprises a round cross-section.

8. The system of claim 6, wherein the cut-out is an arced cut-out.

9. The system of claim 6, wherein said cutting member comprises at least one of a single vertical blade and a double horizontal blade.

10. The system of claim 6, the cutting member housing having a first end and a second end.

11. The system of claim 10, further comprising a second mounting stem.

12. The system of claim 11, the second mounting stem coupled to the drive mechanism and the second end of the cutting member housing, and the mounting stem coupled to the drive mechanism and the first end of the cutting member housing.

13. The system of claim 6, the axis of rotation being a vertical axis of rotation.

14. The system of claim 6, wherein the cutting member is disposable.

15. A method for cutting a medical tube, the method comprising:
selecting a cutting device, the cutting device comprising:
a blade housing, a blade connected to the blade housing, the blade housing having a first end and a second end, and the blade having an axis of rotation;
a first mounting stem coupled to the first end of the blade housing, a second mounting stem coupled to the second end of the blade housing; and
a cradle plate positioned proximal to the blade, the cradle plate comprising a circular passageway extending therethrough perpendicular to the axis of rotation of the blade, and an arced cut-out in the axis of rotation of the blade, the blade receivable within the arced cut-out;
coupling the medical tube to a gripper;
positioning the medical tube within the circular passageway of the cradle plate; and
cutting the medical tube by rotating the blade axially into the arced cut-out of the cradle plate.

16. The method of claim 15, wherein the step of rotating the blade axially into the arced cut-out of the cradle plate comprises rotating the blade at least 180 degrees, the blade thereby cutting an arc-shaped cut out in the medical tube.

17. The method of claim 15, wherein the step of cutting the medical tubing by rotating the blade housing axially into the arced cut-out of the cradle plate comprises:
rotating the blade into the arced cut-out;
the gripper moving the medical tube a length; and
rotating the blade out of the arced cut-out, the blade thereby cutting a slotted opening in the medical tube.
